(19) United States
(12) Patent Application Publication
(10) Pub. No.: US 2003/0044456 A1
(43) Pub. Date: Mar. 6, 2003

(54) Hypoallergenic gelatin

(75) Inventors: Kenichi Ichie, Tsukuba-shi (JP); Yasuki Taguchi, Tsukuba-shi (JP); Yoshihisa Takahata, Tsukuba-shi (JP); Fumiki Morimatsu, Tsukuba-shi (JP); Tamotsu Shigehisa, Tsukuba-shi (JP)

Correspondence Address: BIRCH STEWART KOLASCH & BIRCH, PO BOX 747, FALLS CHURCH, VA, 22040-0747, US

(73) Assignee: Nippon Meat Packers, Inc., Osaka (JP)

(21) Appl. No.: 10/270,607

(22) Filed: Oct. 16, 2002

Related U.S. Application Data
(63) Continuation of application No. 09/830,056, filed on Jul. 24, 2001.
Application No. 09/830,056 is a 371 of international application No. PCT/JP99/05890, filed on Oct. 25, 1999.

(30) Foreign Application Priority Data
Oct. 23, 1998 (JP) 10-319929

(51) Int. Cl. A61K 38/17; A61K 9/64; C07K 14/78; A23J 1/02; A23J 1/04

(52) U.S. Cl. 424/456; 530/354; 426/657

(57) ABSTRACT

This invention provides gelatin and gelatin hydrolysate peptide for food, medical and cosmetic use derived from such raw materials as poultry skins, bones and/or tendons, with an isoelectric point at pH 7-10, and characterized by no or low antigen-antibody reactivity to the serum of gelatin-allergic individuals. Since the gelatin and its hydrolysate peptide of the invention are hypoallergenic, they can safely be used for gelatin-allergic individuals and for food, medicine and cosmetics, for which the safety is particularly required.

### TECHNICAL FIELD

This invention relates to hypoallergenic gelatin and peptide. More particularly, the invention relates to gelatin and peptide utilizable for food, medicine (including drugs) and cosmetics, causing no allergic symptoms.

### PRIOR ARTS

Gelatin is produced by physicochemical denaturation and solubilization of collagen, a major constituent protein of vertebrate animals, followed by purification and dehydration. Therefore, gelatin is produced from such collagen-containing tissues as skins, bones, cartilage, tendons and placentas of animals (e.g., bovine, swine, rabbits, sheep and poultry). To produce gelatin commercially, bovine and porcine bones and skins are used because of their large supply at a low cost.

To produce gelatin, raw materials are usually pretreated with alkali or add and extracted with hot water. Recently, enzymatic (protease) pretreatment has also been adopted to improve the efficiency of production and the purity of the product.

For alkaline pretreatment, raw materials are soaked in 1-5% lime-water for 30-100 days and then washed for neutralization. Such processing removes impurities and makes gelatin easily extractable with hot water. Since amide groups of arginine, gultamic acid and aspartic acid residues of gelatin are carboxylated during alkaline processing, the isoelectric point of alkali-pretreatment gelatin is at pH 4-5.5, being lower than that of collagen.

For acid pretreatment, raw materials are soaked in dilute sulfuric acid or hydrochloric acid for 1-2 days, followed by washing. The isoelectric point of acid gelatin is at pH 7-10, usually at pH 7.5-9.5, being close to that of collagen. That of enzymatically-pretreated gelatin is also close to that of acid-pretreated gelatin.

Because of various characteristics such as foaming, film-forming, water-holding, colloid-protecting, elastic and heat-reversible sol-gel converting (gelling) and availability in large quantities at a low price, gelatin is extensively utilized in the food, medical, cosmetic, photographic, and industrial fields.

By making use of the gelling property, gelatin can be used as a raw material for jelly Although such plant polysaccharides as agar, carrageenan and pectin also have a gelling property, they are not digestive, absorbable nor nutritious. Gelatin is one of the most digestive and absorbable animal proteins and gives palatability to jelly. Because of the foaming property, gelatin can be used as a raw material for marshmallow. Besides, it is widely used as main or subsidiary materials for gummy candy, yogurt, hams, sausages, soup, bavaroise, and ice cream.

Depending on the viscous, gelling, and film-forming properties, gelatin is used to produce hard and soft medical capsules. Making use of the affinity to the skin and water-retaining and viscous properties, gelatin is used in poultices and also as a binder of tablets and in hemostats. For cosmetic use, gelatin is added to milky lotions, packs, and the like as a moisture-retaining component.

In photographic industry, gelatin is used as a binder of silver halogenide, a photosensitive reagent. With regard to the gelling and colloid-protecting properties, no substitute for gelatin has ever been developed. In manufacturing industry, gelatin is used as an adhesive of plywood, furniture and the like.

Gelatin hydrolysate peptide produced with acid, alkali, enzyme or heat treatment is also widely used for food, medicine and cosmetics. Since gelatin hydrolysate is more soluble and absorbable than untreated gelatin, it is added to nutrient supplements as an amino acid source. Depending on the colloid-protecting property, gelatin hydrolysate is used as a dreg-precipitating substance to produce such alcoholic beverages as Sake. It is used as plasma substitute, vaccine stabilizers and the like in the medical field and added to the materials of hair-care goods and the like in the cosmetic field.

Since there is little difference in the molecular structures of gelatin derived from different species of animals, gelatin has been regarded as having no or little antigenicity. The safety of food gelatin to man has widely been accepted, since FAO/WHO Joint Expert Committee on Food Additives categorized food gelatin as “category A (1), unnecessary to specify ADI”. Gelatin is also accepted as a safe pharmacological ingredient, as it is listed in the third edition of Japanese Pharmacopoeia.

Recently, however, such infants suffering from allergic reactions after giving gelatin-containing vaccines or ingestion of food are increasing in number. One out of approximately 500 and 110 infants are suffering from immediate- and delayed-type gelatin allergy, respectively (the Research Group on Side Effects of Vaccination, the Vaccination Research Center, the Ministry of Health and Welfare of Japan; Studies on efficient vaccination and its side effects, 151-154, 1998). Triple vaccine, gelatin-containing weaning foods and the like have been ascribed to such sensitization.

Although gelatin is widely used in food, medical, cosmetic, photographic and industrial products, administration of such products to gelatin-allergic persons may provoke allergic symptoms. Severe gelatin allergy is caused by inoculation of live vaccine. To prevent reduction in the live-vaccine titer, addition of such a proteinous stabilizer as gelatin, its hydrolysate or human serum albumin has been indispensable. However, use of human serum albumin may be hazardous because of the possible presence of unknown pathogens. The stabilizing activities of such saccharides as lactose, sorbitol, glucose, sucrose and dextran are low. Recently, some gelatin-free vaccines have been developed in view of well-known hazard of gelatin-containing vaccines. However, addition of a protein stabilizer to measles virus vaccine is still necessary, since it requires to exhibit stable activities at low titers. Consequently, development of substitutes for functional stabilizers or gelatin causing no allergic symptoms has strongly been desired.

Although gelatin allergy due to vaccination has been recognized, little attention has been paid to allergy caused by food, medicine or cosmetics. It is anticipated that these products may possibly cause severe accidents; therefore, prevention of such accidents seems to be socially very important.

There are many kinds of gelatin-containing food eaten everyday, including jelly, bavaroise, gummy candy, yogurt, hams, sausages, soup, and ice cream. Gelatin hydrolysate peptide is also added to food; particularly, beverages containing a large amount of gelatin hydrolysate peptide have recently been marketed. The hydrolysate peptide contents of such food sometimes exceed 5 g per portion, which is sufficient to cause anaphylaxis shock. For the main material for hard and soft medical capsules, gelatin is also used. By use of such food or drugs, gelatin-allergic patients may develop such allergic symptoms as itch in their mouths and eyes, runny nose, fever, vomiting, headache, diarrhea, skin inflammation, asthma and, in some cases, even anaphylaxis shock. As described above, gelatin is an indispensable ingredient to maintain the shape, texture and palatability of food and drugs, nevertheless no inexpensive substitute has ever been developed.

Gelatin is added to milky and beauty lotions as a moisture-retaining component and used also in poultices. If gelatin-allergic patients use such cosmetics or poultices, they may develop such skin inflammation as itch, erythema, swelling, blister, and exfoliation. However, no low cost substitute with such properties as skin affinity, moisture retention and viscosity has ever been developed.

As described above, use of gelatin in food, medicine, and cosmetics depends on its various properties, most of which cannot be substituted by any other substance. Consequently, development of inexpensive substances with not only the same properties as those of conventional gelatin but also safety causing no allergy has been desired.

From such a viewpoint, hypoallergenic gelatin has been developed from low antigellic type-It collagen (see Japanese Patent Application No. 210075/1997). It is, however, difficult to obtain large quantities of such materials as cartilage containing type-II collagen. Cartilage is more expensive than bovine or porcine skin and bones and unsuitable for commercial production of gelatin because of poor recovery.

Besides the above-described invention, various other inventions including hydrolysis of gelatin to remove allergenic property have been proposed (see Japanese Patent Publication No. 36495/1987, Japanese Patent Application Laid-open Nos. 82299/1995, 193958/1996, 17619/1997, etc.). Application of gelatin hydrolysate of such inventions is quite limited, because indigenous properties of gelatin (e.g., gelling and film forming) are destroyed by hydrolysis. As shown in Examples mentioned later, commercially available gelatin preparations derived from bovine or porcine skins or bones and those produced by alkaline processing are allergenic Gelatin hydcrolysate produced according to the above-described patent gazettes may retain allergenicity.

Consequently, the previous inventions do not deal with gelatin or its producing method the invention aimed at, namely having the same properties as those of conventional gelatin, and yet being nonallergenic, safe, and low price. To solve the above problems, the present inventors attempted to develop a method to quantitatively and economically produce gelatin having the same processing properties and palatability as conventional gelatin, no or low antigen-antibody reactivity to the serum of gelatin-allergic patients, and the safety. The inventors succeeded in finding that gelatin produced from the skins, bones and/or tendons of poultry, preferably chicken, without alkaline treatment could meet the expected purposes. This invention was accomplished on the basis of such findings and can economically and quantitatively provide gelatin causing no allergy to gelatin-allergic individuals and applicable to food, medicine and cosmetics.

### DISCLOSURE OF THE INVENTION

This invention provides the gelatin to be used for food, medicine and cosmetics derived from such raw materials as poultry skin, bones and/or tendons. It has an isoelectric point at pH 7-10 and is characterized by no or lower antigen-antibody reactivity to the serum of gelatin-allergic individuals.

This invention deals also with food, medicine and cosmetics containing the above-described gelatin, being hypoallergenic to gelatin-allergic individuals.

Moreover, the invention includes the methods to produce the above-described gelatin by soaking poultry skins, bones or tendons in an acid solution, recovering and heat-extracting the precipitate, and purifying gelatin from the extract.

This invention includes also peptide for food, medical and cosmetic use, obtained by hydrolysis of the above-described gelatin and/or its precursor, collagen.

### BRIEF DESCRIPTION OF DRAWINGS

FIG. 1 shows the IgE-antibody titers of serum samples of gelatin-allergic individuals (the closed columns) and healthy donors (the open ones) against the gelatin (chicken foot and skin gelatin) and the gelatin-hydrolysate peptide (chicken foot gelatin hyclrolysate) of the invention, and commercially available gelatin for food, medical and cosmetic uses and gelatin hydrolysate (hydrolysate of medical gelatin).

FIG. 2 illustrates the antigen-specific IgE-antibody titers of the serum samples of gelatin-allergic patients against acid-(the closed column) and alkaline-processed gelatin (the open one) from chicken feet and bovine and porcine skins

### THE BEST MODE FOR CARRING OUT THE INVENTION

As described above, the gelatin of the invention is derived from such raw materials as poultry skins, bones and/or tendons, with an isoelectric point at pH 7-10, and characterized by little antigen-antibody reactivity to the serum of gelatin-allergic individuals who recognize conventional gelatin as an allergen. The above-described low antigen-antibody reactivity is defined as giving no statistically significant difference in the antigen-antibody reactivity to the serum of gelatin-allergic individuals from that to the serum of healthy donors.

Poultry skins, bones and/or tendons are used as raw materials for the hypoallergenic gelatin of the invention. By-products of meat industry can be used as-raw materials. The poultry comprise the chicken, duck, goose, turkey and the like; the chicken furnishes a favorable raw material. Therefore, chicken skins, bones and/or feet are favorably used in the invention. The source of the gelatin of the invention can be confirmed by antigen-antibody reaction.

The gelatin of the invention is produced from above-described raw materials, but it is important not to use alkaline processing in the production. Gelatin products on the market are mostly pretreated with alkali, which increases the allergenicity of the product regardless of the animal species or the organs as indicated in Examples mentioned later. Therefore, sufficient attention must be paid to the pretreatment.

Whether or not the alkaline pretreatment has been applied can be ascertained by examining the isoelectric point of the resulting gelatin;, The isoelectric point of acid-processed gelatin is at pH 7-10, usually at pH 7.5-9.5, being close to that of collagen. That of alkali-pretreated gelatin is at pH 4-5.5, being lower than that of collagen, since the amide groups of arginine, gultamic acid and aspartic acid residues are carboxylated.

Beside the isoelectric point, application of alkaline pretreatment can be ascertained by detecting ornithine by amino acid analysis. Alkaline processing partly changes arginine to ornithine. Such a change can be confirmed by amino acid analysis, since a few ornithine residues can be detected among 1,000 amino acid residues in alkaline-processed gelatin but not at all in intact collagen or acid-processed gelatin.

An example of the method to produce the gelatin of the invention is more concretely indicated. The above-described raw materials are soaked for two days or so in an acid solution (hydrochloric acid or sulfuric acid, preferably an about 3% hydrochloric acid solution). Then the formed precipitate is recovered, washed and then thermally extracted with an acid solution at pH approximately 4 (hydrochloric acid or sulfuric acid, preferably a hydrochloric-acid solution). The gelatin of the invention is obtained by filtering, desalting and drying the extract. The gelatin thus obtained may be further purified by a conventional method for protein purification, if necessary.

As indicated in Examples mentioned later, the jelly strength of the gelatin of the invention is identical to that of conventional one. As the gelatin of the invention possesses foaming, film-forming, water-holding, colloid-protecting, and elastic properties, it can be applied to the areas similar to those of conventional gelatin. The gelatin of the invention can be applied more favorably to food, medicine and cosmetics.

For food use, the gelatin of the invention can be applied to jelly, gummy candy, yogurt, hams, sausages, soup, bavaroise, ice cream and the like. By using the gelatin of the invention, very little allergenic food can be produced without changing the palatability.

For medical use, the gelatin of the invention can be applied to capsules, poultices, vaccine stabilizers and the like. By using the gelatin of the invention, very safe products can be produced without changing the basic properties of conventional gelatin In vaccine production, the gelatin of the invention can be used to detoxify the toxin.

For cosmetic use, the gelatin of the invention can be added to cream, ointment, beauty lotion and the like. As the gelatin of the invention is no or less allergenic, it helps prevent skin inflammation caused by cosmetics containing conventional gelatin.

As far as hypoallergenic property is ensured, the gelatin of the invention can be mixed with another kind of gelatin (e.g., alkaline-processed gelatin, gelatin from animals other than poultry and the like). Such mixing helps maintain processing properties and/or palatability and provides less costly products.

The gelatin hydrolysate peptide of the invention contains low molecular-weight substance prepared by hydrolysis of the above-described gelatin and is characterized by no or low antibody-antigen reactivity to the serum of gelatin-allergic individuals, recognizing conventional gelatin from bovine or porcine bones and skins as an allergen.

The gelatin hydrolysate peptide of the invention can be produced by hydrolysis of gelatin of the invention and/or its precursor, collagen, with an enzyme or acid. Although the molecular weight can be adjusted according to the use, the gelatin hydrolysate with that ranging from 500 to 50,000 may favorably be used.

As it possesses not only the fundamental properties of hydrolysate of conventional gelatin or collagen but also safety, the gelatin hydrolysate peptide of the invention is applicable to the same use areas as hydrolysate of conventional gelatin or collagen. Particularly, as the low-molecular weight hydrolysate is highly soluble in water but does not jell even at low temperatures, it can be used for such food as beverage and confectionery, such medicine as vaccine stabilizers, poultices and plasma substitutes, and such cosmetics as shampoo.

### INDUSTRIAL APPLICABILITY

This invention provides gelatin and gelatin hydrolysate peptide possessing not only the same properties of ordinary ones but also hypo-allergenic property. Therefore, they are useful for food, medicine, cosmetics and the like, which particularly require safety. The gelatin and its hydrolysate peptide of the invention can utilize by-products of meat industry such as poultry skins, bones and tendons. Therefore, it is possible to make best use of natural resources and to produce a large quantity at low cost because of their high yield.

### EXAMPLES

This invention will specifically be explained in detail with Examples and Experiments, but the scope of the invention is not restricted to these Examples.

### Example 1

Preparation of Hygoallergenic Gelatin (1)

Chicken feet were ground and soaked in 5 volumes of a 3% hydrochloric acid solution. The formed precipitate was recovered 48 h later, washed to remove excessive acid, and extracted with 5 volumes of a hydrochloric acid solution (pH 4) for 4 h at 60° C. The extract was passed through a mixed bed-type ion exchange resin column (BioRad, AG501-X8) previously washed with distilled water. The pass-through fraction was recovered and lyophilized, to obtain hypoallergenic gelatin of the invention.

### Example 2

Preparation of Gummy Candy

Yogurt powder (6 weight % of the final mixture, likewise in the following) was dissolved in water (14%). Then, sugar (25%), millet-jelly (30%) and the hypoallergenic gelatin prepared in Example 1 (25%) were added and boiled down for 20 min. The mixture was stuffed into an appropriate casing, formed and cooled to obtain gummy candy.

### Example 3

Preparation of Jelly

Hypoallergenic gelatin prepared in Example 1 (3%) and sugar (10%) were dissolved in hot water (85%). Then, fresh lemon juice (2%) was added and boiled for 20 min. The mixture was formed and cooled to obtain jelly.

### Example 4

Preparation of Hard Capsule

A 5% solution of hypoallergenic gelatin prepared in Example 1 was attached on capsule-shaped pins, cooled and dried. The pins were removed, when the moisture content reduced to 15-18%. Dehydration was continued to attain the moisture content of 12-15% to obtain hard capsules.

### Example 5

Preparation of Hypoallergenic Gelatin (2)

Hypoallergenic gelatin of the invention was prepared in the same manner as Example 1 except for using chicken skin in place of chicken feet.

### Example 6

Preparation of Hypoallergenic Gelatin Hydrolysate

In 500 ml of 3% hydrochloric acid, 10 g of hypoallergenic gelatin prepared in Example 1 was dissolved and hydrolyzed for 3 h at 70° C. After neutralization, the hydrolysate was passed through a mixed bed-type ion exchange resin column (BioRad, AG501-X8) previously washed with distilled water. The pass-through fraction was recovered and spray-dried to obtain the gelatin hydrolysate peptide of the invention,

### Example 7

Preparation of Fruit Juice

Citric acid (0.2%), orange juice (35%), sugar (5%) and gelatin hydrolysate peptide prepared in Example 6 (3%) were dissolved in water (56.8%), heated, cooled and packaged. After heat treatment for 30 min at 85° C., fruit juice was obtained.

### Example 8

Preparation of the Hypoallergenic Gelatin (3)

Hypoallergenic gelatin of the invention was obtained in the same manner as Example 1 except for using duck skins in place of chicken feet.

Experiment I

Demonstration of Hygoallergenic Property (1)

The activities of antigen-specific IgE antibodies ill the serum of gelatin-allergic individuals against the gelatin and gelatin hydrolysate peptide of the invention prepared in Examples 1, 5 and 6, and commercially available gelatin were examined.

\mathring{1} Test Samples

The gelatin and gelatin hydrolysate peptide of the invention, commercially available gelatin for food, medicine and cosmetics and gelatin hydrolysate for medical use were used as test samples. The characteristics of these samples are listed in Table 1. Jelly strength was determined by the method described in Japanese Industrial Standard JIS K 6503-1977.

TABLE 1

| | | Jelly strength |
|---|---|---|
| Test sample | Raw material | (Bloom) |
| Chicken-foot gelatin (Example | Chicken feet | 414 |
| 1) | | |
| Chicken-skin gelatin (Example | Chicken skins | 375 |
| 5) | | |
| Chicken-foot gelatin- | Chicken feet | Not gelled |
| hydrolysate peptide (Example | | |
| 6) | | |
| Food gelatin commercially | Bovine skins | 100-300 |
| available | | |
| Medical gelatin commercially | Bovine bones | 284 |
| available | | |
| Cosmetic gelatin commercially | Bovine bones, skins | Not gelled |
| available | | |
| Medical gelatin hydrolysate | Bovine bones, skins | Not gelled |
| commercially available | | |

\mathring{2} Preparation of Test Sample Solutions

The gelatin and gelatin hydrolysate peptide of the invention and commercially available gelatin were each dissolved in PBS (phosphate-buffered saline, pH 7.2) with constant stirring to prepare a 0.1% (w/v) test solution.

\mathring{3} The Serum of Gelatin-Allergic Individuals

Blood samples were collected from 15 RAST (radio allergosorbent test)-positive gelatin-allergic individuals (six boys and nine girls) by a physician. The serum samples conventionally prepared were kept frozen until used.

\mathring{4} Serum Samples of Healthy Individuals

Umbilical blood samples were collected from six healthy neonates. The serum samples conventionally prepared were kept frozen until used.

\mathring{5} Experimental Methods

The activities of antigen specific-IgE antibodies in the serum of gelatin-allergic individuals against the above-described test solutions (antigen-specific IgE-antibody titers) were determined by ELISA (enzyme-linked immunosorbent assay). ELISA was carried out according to the method of O. Fujiwara et al (Eds.), Immunology Research Handbook, pp. 199-206, 1992. Chugai Igakusha, Tokyo.

The outline of ELISA was as follows:

(1) The test solutions were placed on a 96-well microplate and the antigens were immobilized.

(2) After washing the microplate, the test samples and a labeled antibody were blocked by adding human serum albumin to prevent non-specific binding.

(3) After washing the microplate, the sera from allergic and healthy individuals were each added to the well to allow to react with the immobilized antigen.

(4) After washing the microplate, alkaline phosphatase-labelled anti-human IgE ε-chain goat antibody was added.

(5) After washing the microplate, a substrate (Lumiphos 530; 4-methoxy-4-(3-phosphate-phenyl) spiro [1,2-dioxetane-3.2°-adamatane] disodium salt, Wako Junyaku Kogyo) was added. Photons emitted by alkaline phosphatase dephosphorization were determined.

(6) The photons were determined with a plate reader (Luminous CT-9000D, Diatron). The antigen-specific IgE-antibody titers were calculated according to a calibration curve previously prepared with serially diluted standard antibody.

\mathring{6} Results

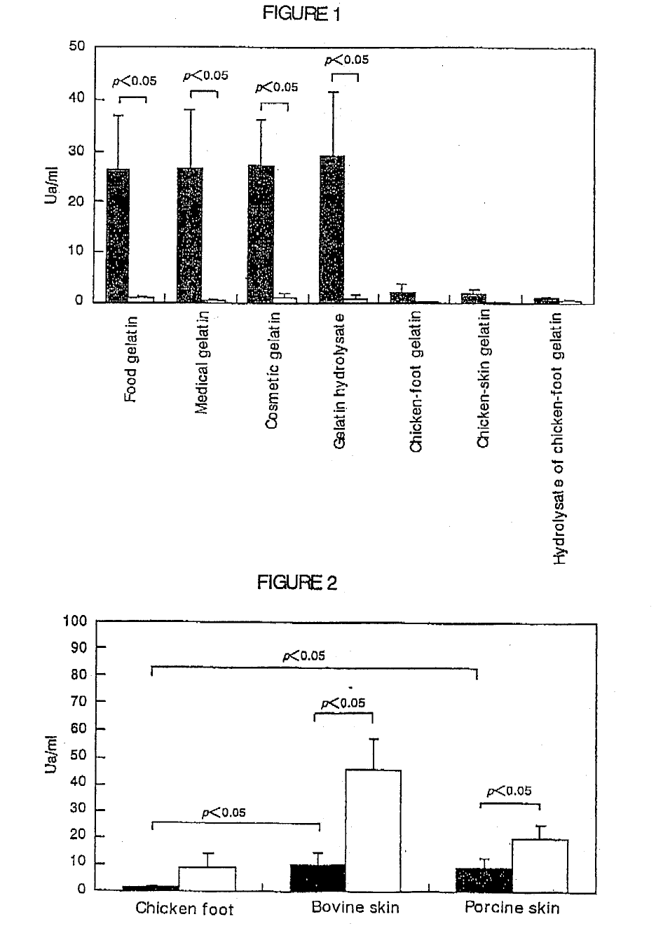

FIG. 1 illustrates the results of the above tests (mean standard error). The closed and open columns indicate the antigen-specific IgE-antibody titers against the test samples in the sera from gelatin-allergic and healthy donors, respectively.

As shown in FIG. 1, the antigen-specific IgE-antibody titers of both sera against the gelatin and its hydrolysate peptide of the invention were low. However, the antigen-specific IgE-antibody titers against commercially available food, medical and cosmetic gelatin preparations and medical gelatin-hydrolysate were high in the serum of gelatin-allergic patients but significantly lower in healthy individuals.

Experiment 2

Demonstration of Hypoallergenic Property (2)

To study the differences in allergenic properties of various animal sources and the processing methods, acid- or alkaline-processed gelatin was prepared. The antigen-specific IgE-antibody titers of the serum of gelatin-allergic individuals against these gelatin preparations were examined. Acid-processed gelatin from chicken feet was identical to the gelatin of the invention described in Example 1.

\mathring{1} Test Samples

Acid or alkaline-processed gelatin conventionally prepared from chicken feet and bovine and porcine skins were used as test samples. The number of ornithine residues (per 1,000 amino acid residues) and isoelectric points of the samples are listed in Table 2.

TABLE 2

| | Number of ornithine | |
|---|---|---|
| | residues (per 1,000 | |
| Test sample | amino acid residues) | Isoelectric point |
| Acid-processed gelatin from | 0 | 7.5-9.5 |
| chicken feet | | |
| Alkaline-processed gelatin | 3 | 4-5 |
| from chicken feet | | |
| Acid-processed gelatin from | 0 | 7-9 |
| bovine skins | | |
| Alkaline-processed gelatin | 10 | 4.5-5 |
| from bovine skins | | |
| Acid-processed gelatin from | 0 | 7-9 |
| porcine skins | | |
| Alkaline-processed gelatin | 9 | 4-5 |
| from porcine skins | | |

\mathring{2} Preparation of Test Sample Solutions

Test sample solutions were prepared in the same manner as described in Experiment 1.

\mathring{3} Serum Samples of Gelatin-Allergic Individuals

The same serum samples as described in Experiment 1 were used.

\mathring{4} Experimental Methods

Examination was carried out in the same manner as described in Experiment 1.

\mathring{5} Results

FIG. 2 illustrates the results of the above tests (mean±standard error). The dosed and open columns indicate the antigen-specific IgE-antibody titers of the serum samples of gelatin-allergic individuals against acid- and alkaline-processed gelatin preparations, respectively. As shown in FIG. 2, the IgE-antibody titers against the gelatin of the invention were low, but those against acid-processed gelatin from porcine and bovine skins were significantly high.

Regardless of the raw materials, the alkaline processing increased the IgE-antibody titer in the serum samples of gelatin-allergic individuals. From the test separately carried out, the IgE-antibody titers against the gelatin of the invention in the serum samples of gelatin-allergic individuals were not significantly different from those of healthy ones. Namely, the IgE antibodies specifically recognizing the gelatin of the invention as an antigen (allergen) were scarcely found in the serum samples of gelatin-allergic individuals. However, the IgE antibodies against alkaline-processed gelatin from chicken feet, the raw material of the invention, were found in the serum of gelatin-allergic individuals. As for gelatin derived from bovine and porcine skins, an allergenic property was observed regardless of the processing methods.

The reason why the allergenic property of alkaline-processed gelatin is higher than that of acid-processed one may depend on the altered primary structure of the gelatin molecule and the decreased sequence similarity to human collagen (gelatin) caused by alkaline processing.

Although the data are not shown, similar results as those with commercial gelatin were obtained with commercial gummy candies, jelly and hard capsules. Gummy candies, jelly, hard capsules and fruit juice of Examples 2, 3, 4 and 7, containing the gelatin or the gelatin hydrolysate peptide of the invention hardly reacted with the serum of gelatin-allergic individuals.

From these results, gelatin-allergic individuals should avoid food, medical or cosmetic gelatin preparations and medical gelatin-hydrolysate commercially available because of their high IgE-antibody activities against these products. The gelatin of the invention is characterized by a low reactivity to the antigen-specific IgE antibody and its safety. Even gelatin-allergic individuals can safely eat gummy candies, jelly, hard capsules, fruit juice and the like prepared from the gelatin or its hydrolysate peptide of the invention.

With regard to commercially available gelatin hydrolysate peptide, reduction in the allergenic property had been expected due to the reduced molecular size, but reduction in the allergenic property was obviously insufficient as shown in the results of the above-described test. The gelatin hydrolysate peptide of the invention, however, can effectively prevent allergy, since it is prepared by hydrolysis of the hypoallergenic gelatin of the invention.

Experiment 3

Demonstration of Safety (in vivo Examination)

To confirm the safety of gummy candy prepared by the method of Example 2, a loading test to the individuals slightly allergic to gelatin was carried out in collaboration with a medical facility. The loading test was performed according to S. Kaminogawa et al. (Eds.), Food-allergy Countermeasure Handbook, pp. 143-150, 1996, Science Forum, Tokyo.

\mathring{1} Individuals Subjected

Four infants (two boys and two girls, 1-5 year old), who had shown allergic symptoms from eating gelatin-containing food, were subjected to the test. After explaining the aims and details of the test in advance, informed consents were obtained from their legal representatives, whenever possible, or from the individuals themselves. Such patients applicable to the following criteria were excluded from the test.

The patients given reaction-modifying steroids, anti-histamines, anti-allergic medicines and the like.

The patients whose symptoms were not improved despite of exclusion of gelatin, or those whose gelatin-exclusion periods did not exceed one month.

The patients who were likely to develop anaphylaxis shock or acute asthma.

The patients whose participation in the test was judged to be improper by their physicians.

\mathring{2} Test Food

Gummy candy was prepared by the method described in Example 2, formed in a size of 2.5 g per piece, and supplied as test food.

\mathring{3} Method of Administration

All the patients were hospitalized and their vein routs were secured to cope with in case of anaphylaxis shock. The test food was administered to the patients at their hunger and with the presence of their legal representatives. According to the gelatin-specific IgE-antibody titers of the patients, their rations were determined and the single-blind test was performed. Although the maximum dose was fixed to 10 pieces of candy, administration was started with a small dose (one piece) If no side reaction was observed, administration was repeated in every 15 min with increasing doses and completed in an hour. Whenever any symptom was observed, the loading test was stopped and the following course was observed.

\mathring{4} Methods of Observation

Changes of the skin, digestive and respiratory organs and anaphylaxis were observed before, at start, and 30 min, 1, 2, 3, 5, 24 and 48 h after the administration.

\mathring{5} Items to be Evaluated

Evaluation was made by the physicians on the following three criteria:

(1) Did the test food cause allergic reaction in the patients? (Diagnosis for positive reaction)

(2) How and where on the patients' bodies did the symptoms appear? (Diagnosis for pathological changes)

(3) How long did it take for the symptoms to appear after the first administration? (Diagnosis for the time to symptom appearing)

\mathring{6} Judgement

From the physicians' overall evaluation, the safety of the test food was categorized into three: ‘not problematic at all’, ‘somewhat problematic’ and ‘surely problematic’.

\mathring{7} Results

The results of the loading test are shown in Table 3. As an overall evaluation, the test food was ‘not problematic at all’ and ‘somewhat problematic’ in three and one of the four subjects, respectively. The latter patient developed urticaria after eating a piece of gummy candy commercially available, but he did not do so after eating one of the invention. He developed mild urticaria on his back in 30 min after eating 10 pieces of gummy candy.

TABLE 3

| Rate of positive results | One case/four patients |
|---|---|
| Symptoms developed on the | Mild urticaria in one case (on the back |
| skin | in 30 min after administration) |
| Symptom developed on the di- | None observed |
| gestive tract | |
| Symptoms developed on the | None observed |
| respiratory organ | |
| Anaphylaxis | None observed |

From these results, the present test food can be regarded as a substitute which gelatin-allergic patients can safely and palatably eat.

1. Gelatin for food, medicine and cosmetics derived from such raw materials as poultry skins, bones and/or tendons, with an isoelectric point at pH 7-10, characterized by hypoallergenic property to the serum of gelatin-allergic patients.

2. Gelatin for food, medicine and cosmetics of claim 1 which is prepared by acid treatment of poultry skins, bones and/or tendons.

3. Food containing the gelatin of claim 1 which is hypoallergenic to gelatin-allergic patients.

4. Jelly, gummy candy, yogurt, hams, sausages, soup, bavaroise, and ice cream containing the gelatin of claim 1 which are hypoallergenic to gelatin-allergic patients.

5. Cosmetics containing the gelatin of claim 1 which are hypoallergenic to gelatin-allergic patients.

6. Cream, ointment and beauty lotion containing the gelatin of claim 1 which are hypoarlergenic to gelatin-allergic patients.

7. Medicine containing the gelatin of claim 1 which is hypoallergenic to gelatin-allergic patients.

8. Medical capsules, poultices and vaccines containing the gelatin of claim 1 which are hypoallergenic to gelatin-allergic patients.

9. The method to produce the gelatin of Clam 1, which comprises soaking poultry skins, bones or tendons in an acid solution, recovering the precipitate formed, heat-extracting the precipitate with an acid solution, and purifying the extract.

10. The gelatin hydrolysate peptide for food, medicine and cosmetics produced by hydrolysis of the gelatin of claim 1 and/or its precursor collagen.

11. The peptide of claim 10, where said peptide has a food use.

12. The peptide of claim 10, where said peptide has a cosmetic use.

13. The peptide of claim 10, where said peptide has a medical use.

14. The gelatin of claim 1, where said gelatin is mixed with another kind of gelatin.

15. The gelatin of claim 1, where said gelatin is mixed with alkaline-processed gelatin.
